(19) United States
(12) Patent Application Publication
(10) Pub. No.: US 2013/0178836 A1
(43) Pub. Date: Jul. 11, 2013

(54) Device for adjusting the length of infusion tubing

(71) Applicant: Roche Diagnostics International AG, Rotkreuz (CH)

(72) Inventor: David Teutsch, Schuepfen (CH)

(73) Assignee: Roche Diagnostics International AG, Rotkreuz (CH)

(21) Appl. No.: 13/671,892

(22) Filed: Nov. 8, 2012

Related U.S. Application Data
(63) Continuation of application No. PCT/EP2011/002234, filed on May 5, 2011.

(30) Foreign Application Priority Data
May 12, 2010 (EP) 10162692.7

(51) Int. Cl. A61M 39/08; A61M 39/10; A61M 5/14

(52) U.S. Cl. CPC A61M 39/08; A61M 5/14; A61M 39/10
USPC 604/533; 242/382

(57) ABSTRACT

A device for storing and dispensing infusion tubing is presented. The device comprises a casing, a spool rotatably mounted on the casing for storing the infusion tubing, and a locking structure for positively locking a central portion of infusion tubing on the spool. A locking mechanism locks a rotation of the spool in regard to the casing in at least one direction.

### CROSS-REFERENCE TO RELATED APPLICATIONS

This application is a continuation of PCT/EP2011/002234, filed May 5, 2011, which is based on and claims priority to EP 10162692.7, filed May 12, 2010, which is hereby incorporated by reference.

### BACKGROUND

The present disclosure generally relates to devices for adjusting the length of infusion tubing and, in particular, to devices for adjusting the length of infusion tubing with a casing and a spool rotatably mounted on the casing for winding up the tubing.

Infusion pump devices are a modern and convenient way to deliver liquid medication to patients by injection or infusion. Infusion pump devices can be particularly useful in diabetes therapy and pain therapy where patients need small doses of medication on a regular basis. Modern infusion devices are small enough to be carried concealed on the body, a feature much appreciated by patients who are otherwise healthy to allow them to go about their normal daily activities.

Depending on the type of infusion pump device, flexible infusion tubing is typically used to fluidly connect the infusion pump device, located on the body of the patient, with the infusion site where the liquid medicine is conveyed into the body of the patient usually through a needle, or cannula, penetrating the skin.

The shortest distance between the pump and the infusion site defines the minimum length of the infusion tubing needed. However, infusion tubing is often available only in standardized length units such as, for example, one meter. Excess tubing cannot be removed simply by the user. On one hand, cutting the tubing compromises sterility. On the other hand, typically, connectors are attached at the ends of the tubing for fluidly connecting the tubing to other devices. Furthermore, a user may need to change the position of the infusion pump device during the course of the day. He may, for example, wish to place the infusion pump on the bedside table during sleep.

To avoid the necessity to exchange the tubing several time, the user will typically choose tubing that is sufficiently long enough for all the needs during the normal life of infusion tubing. This also avoids the loss of expensive medication that may remain in the discarded tubing. As a consequence, there is, in most cases, surplus infusion tubing that has to be handled by the user. For example, the user may gather and roll the surplus tubing and affix it in coils with adhesive, for example, on the pump device or on the body. However, depending on the amount, the tubing may be difficult to conceal. Uncoiling the infusion tubing can be also a difficult and time-consuming task depending on the adhesive tape used. Furthermore, there can be a risk of the infusion tubing becoming tangled, kinked or caught on an obstacle.

Generally, the devices known from the prior art have the disadvantage that the retraction and release of the tubing cannot be precisely controlled.

Therefore, there is a need for a device for the easy adjustment of the length of infusion tubing and that can be easily coupled to existing and already mounted infusion tubing, without the need of disconnecting the tubing, and that can store surplus infusion tubing so that the tubing is not damaged, kinked or otherwise compromised and that the device can be inexpensive produced and reliable and easy to use.

### SUMMARY

According to the present disclosure, a device for storing and dispensing infusion tubing is presented. The device can comprise a casing, a spool rotatably mounted on the casing for storing the dispensing infusion tubing, and a locking structure for positively locking a central portion of tubing on the spool by a ratchet-like locking mechanism suitable for locking a rotation of the spool in regard to the casing in at least one direction.

In accordance with one embodiment of the present disclosure, a set comprising an above device and one or more pieces of tubing is presented.

In accordance with another embodiment of the present disclosure, infusion tubing with one or more of the above devices attached to the infusion tubing is presented.

Accordingly, it is a feature of the embodiments of the present disclosure to provide for a device for the easy adjustment of the length of infusion tubing and that can be easily coupled to existing and already mounted infusion tubing, without the need of disconnecting the tubing, and that can store surplus infusion tubing so that the tubing is not damaged, kinked or otherwise compromised and that the device can be inexpensive produced and reliable and easy to use. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIGS. 1a-h illustrate a device with automatic retraction functionality according to an embodiment of the present disclosure.

FIGS. 2a-c illustrate device with automatic retraction functionality according to another embodiment of the present disclosure.

FIGS. 3a-d illustrate detail of the ratchet mechanism of the device in FIGS. 2a-c according to an embodiment of the present disclosure.

FIGS. 4a-c illustrate a manually operated device according to an embodiment of the present disclosure.

FIGS. 5a-c illustrate a manually operated device in which the tubing can be inserted without interrupting the fluid connection according to an embodiment of the present disclosure.

FIGS. 6a-c illustrate a device according to yet another embodiment of the present disclosure.

FIGS. 7a-c illustrate the application of the device shown in FIGS. 6a-c according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

In the context of this specification, the term “locking” does include both an absolute locking, in which a rotation of the spool in regard to the casing is mechanically prevented, as well as a relative locking, which allows rotation of the spool after exceeding a threshold force for releasing the ratchet-like mechanism. In one embodiment with absolute locking, the absolute locking can apply only in one direction, while in the other direction, relative locking, or no locking, may apply.

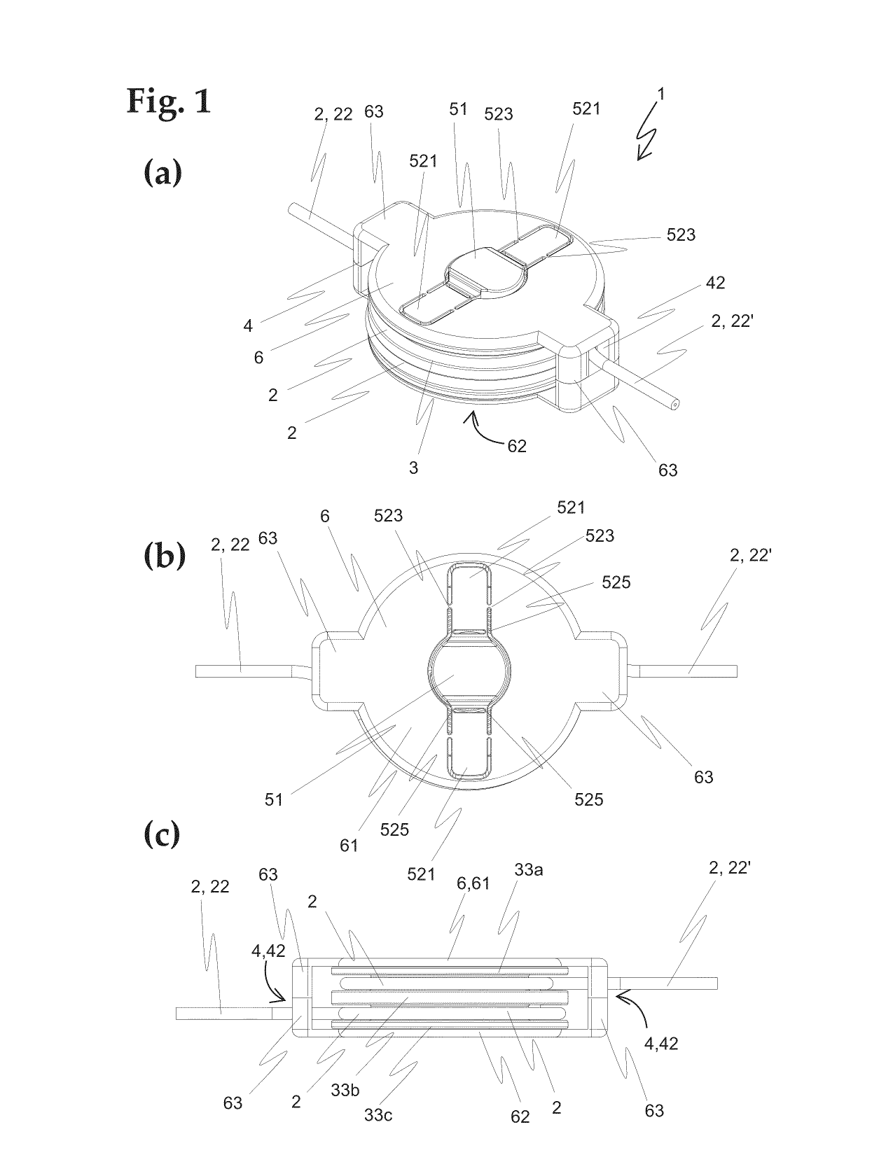

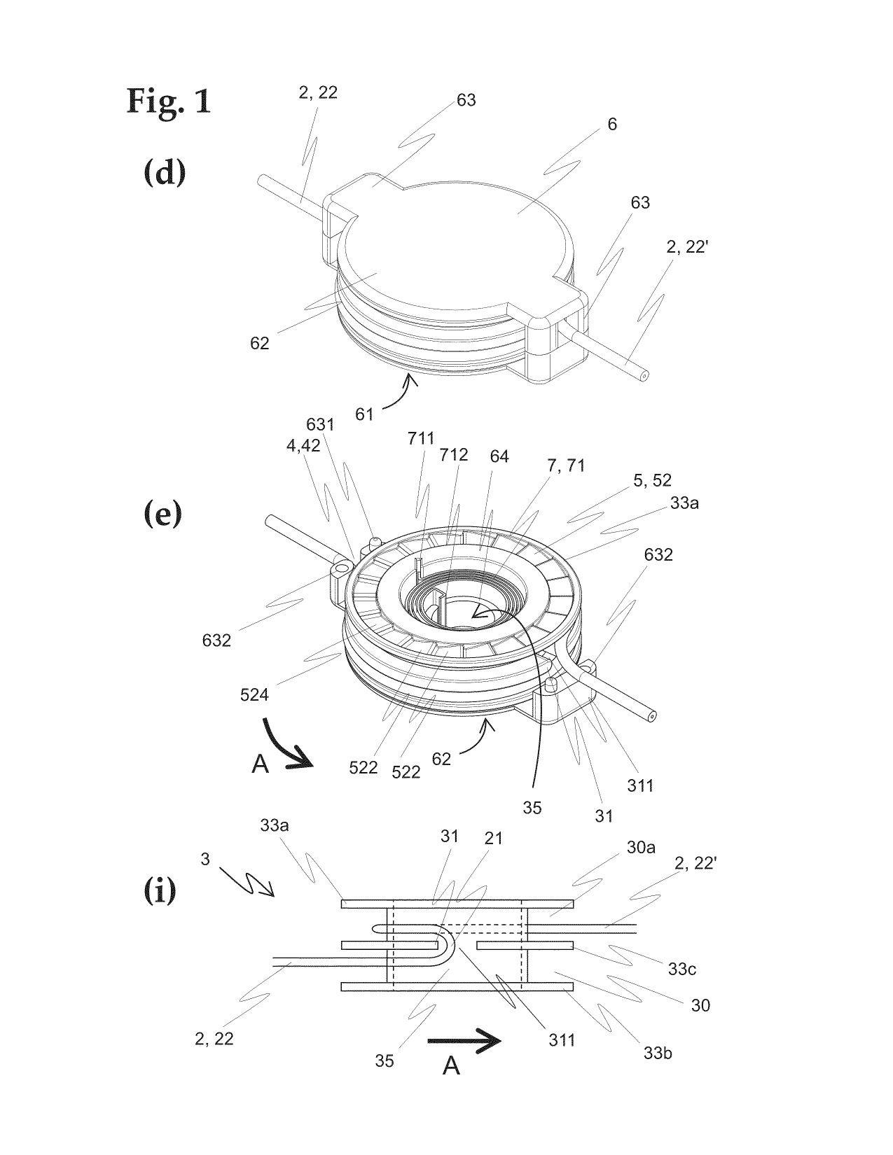

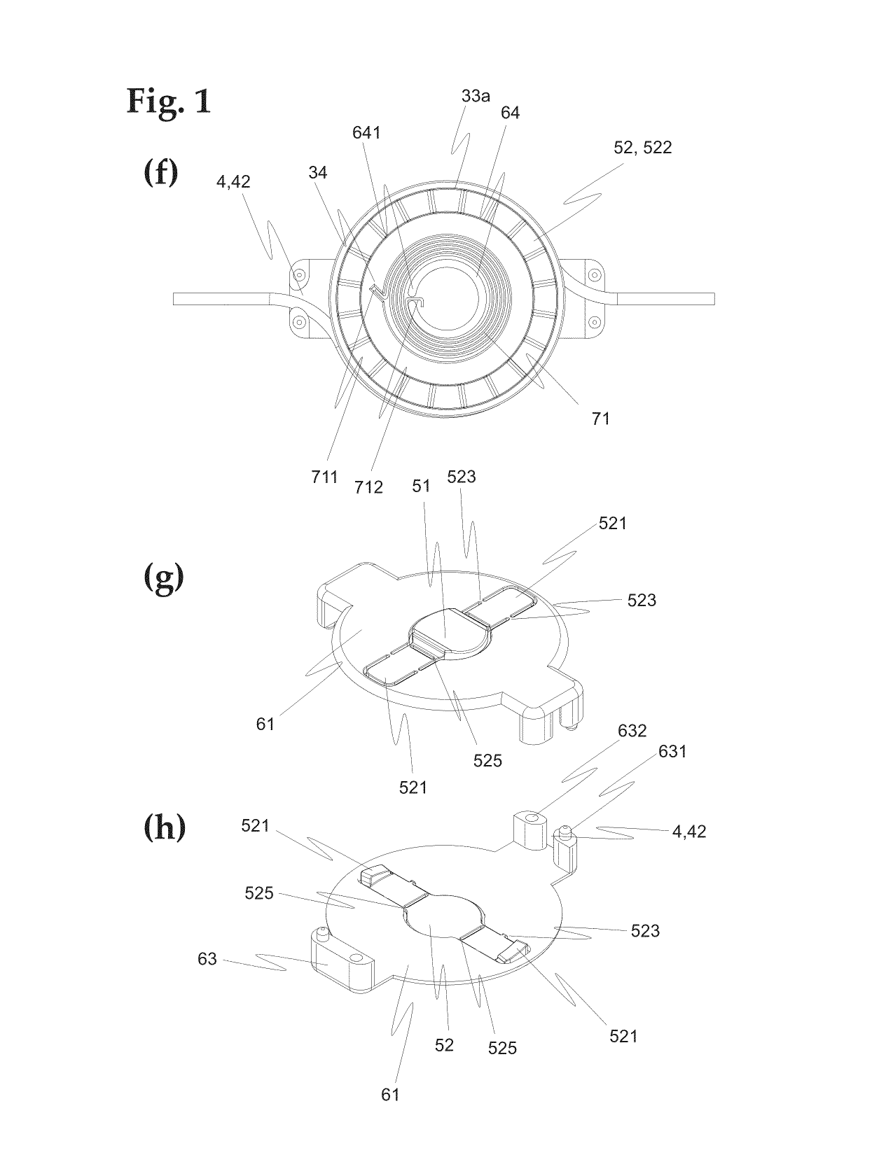

Referring initially to FIG. 1, an embodiment of a device 1 is shown (a) in a perspective view toward the top cover 61, (b) in a top view, (c) in a side view, (d) in a perspective view toward the bottom cover 62, and (e), (f) with removed top cover 61. FIGS. 1(g) and (h) show the removed top cover 61 from both sides. FIG. 1(i) schematically shows a side view of the spool 3, with visible tube locking structure 31.

The embodiment of the device 1 shown in FIG. 1 can comprise a spool 3 that can be rotatably mounted in a casing 6, comprising a top cover 61 and a bottom cover 62. The top cover 61 and the bottom cover 62 can be connected via two bridge structures 63. During use the infusion tubing 2 can be wound onto the spool 3. FIG. 1 shows the device 1 with the tubing 2 already wound up. The tubing 2 can leave the casing 6 via two guiding structures 4, realized as two openings 42 in the bridge structures 63. The guiding structures 4 can direct the tubing 2 toward the spool 3, and can prevent the tubing 2 from unwinding itself when the spool 3 is locked in its rotational movement.

The device 1 can be used in a pre-packed set, with a device 1 provided with the infusion tubing 2 already wound up, in a sterile package. After unpacking the device 1, the user can hold the two ends 22, 22′ of the tubing 2, sticking out of the device 1, and can pull both ends 22,22′ in opposite directions. The tubing 2 can unwind from the device 1, until the necessary amount of tubing 2 is released. The remaining central portion of the tubing 2 can remain coiled on the device 1. One end 22 of the infusion tubing 2 can then be connected with a suitable connector (not shown) to an infusion pump device (not shown) or a similar medical device, or even to a standard IV system. Similarly, the other end 22′ of the tubing 2 can be connected to an infusion cannula (not shown), which has been inserted into the patient's body prior to connecting it to an infusion tubing 2. In other embodiments, the infusion cannula can be realized as an integral part of the tubing 2, in which case, the infusion cannula can be inserted into the patient's body after unwinding the necessary amount of tubing 2 from the device 1.

The device 1 can comprise automatic retraction functionality. By pressing the release button 51 arranged in the center of the top cover 61, two paws 521 of a ratchet mechanism 52 can be temporarily removed from the teeth 522 of a ratchet gear, thereby unlocking the ratchet mechanism 52. A biased spring coil 71 can actuate and rotate the spool 3, rewinding the tubing 2 onto the spool 3. When the button 51 is released, the biased paws 521 can hook into the teeth 522, and the ratchet mechanism 52 can be locked again. When a user pulls both ends 22, 22′ of the infusion tubing 2, the inclined planes of the paws 521 and teeth 522 can glide on each other, according to the standard principle of a ratchet mechanism. A user thus may very easily adjust the length of the tubing 2. He can pull the ends 22, 22′ of the tubing 2 to release the tubing 2 from the device 1 and can retract surplus tubing 2 into the device 1 by pressing the button 51.

The ratchet mechanism 52 is shown in more detail in FIGS. 1(e) to (h). The exterior side of one delimiting flange 33a of the spool 3 can be provided with a circumferential ratchet gear 524 with teeth 522. The spool 3 can comprise a central bore 35, which can be rotatably mounted on a central, hollow hub 64 protruding from the inner side of the bottom cover 62. A spring coil 71 can be arranged in the bore 35 of the spool 3, its first end 711 being positively locked in a slot 34 of the spool 3, and the second end 712 being positively locked in a slot 641 of the hub 64. The teeth 522 of the ratchet gear 524 can interact with two paws 521. The paws 521 can be pivot-mounted on hinge areas 523 and can be connected to the release button 51 via hinge areas 525. Thus paws 521 and release button 51 can be connected with the top cover 62 via the four hinge areas 523.

When the release button 51 is pressed inwards, the paws 521 can be pivoted away from the teeth 522, thereby releasing the ratchet mechanism 52. The deformation of hinge areas 523, 525 can result in a bias, and upon release of the button 51, the paws 521 can be forced back toward the teeth 522, relocking the ratchet mechanism 52. The top cover 62 with paws 521 and button 51 can be manufactured as a single piece by injection moulding. The hinge areas 525 can, for example, be realized as film hinges between paws 521 and button 51. In one embodiment, the hinge areas 523, 525 can be realized as narrow rips.

In one embodiment, the spring coil 71 can be biased in such a way that the spring force is sufficiently strong enough to rewind the tubing 2, but also sufficiently weak enough to be overcome by a user without damaging the tubing 2. Furthermore, the retracting speed should be low in order to give the user precise control over the retraction.

In the device 1, both ends 22, 22′ of the tubing 2 can be retracted at the same time or released at the same time. The device 1 can use a single spool 3, thereby avoiding a complicated gear mechanism and reducing the overall number of parts. As a consequence, the two ends 22, 22′ of the tubing 2 can be wound on the spool 3 in the same direction. For this purpose, a structure 31 can be provided on the spool 3 that can allow positive locking on a central portion 21 of the tubing 2 to the spool 3. When the spool 3 is rotated, both ends 22, 22′ can then be wound in the same direction.

In one embodiment, the spool 3 can be subdivided into two separate spool compartments 30, 30a, by two outer delimiting flanges 33a, 33b and a central delimiting flange 33c. This is schematically shown in more detail in FIG. 1(i). Each end 22, 22′ of the tubing 2 can be wound onto one of the spool compartments 30, 30a. The use of two separate compartments 30, 30a can have the advantage that the two ends 22, 22′ of the tubing 2 cannot mingle with each other during the winding and unwinding procedure.

The central delimiting flange 33c can have one or more radial slots 311 having a width that is broader than the diameter of the tubing 2 to be stored. In a maximum unwound state of the device 1, the tubing 2 will run through a first opening 42 of the guiding structure 4 and along a first spool compartment 30a. It will then pass one of the slots 311 in the central flange 33c into the second compartment 30a, where it reverses its direction, runs along the second spool compartment 30a, and through a second opening 42 out of the casing 6. When the spool 3 is now rotated in one direction of rotation A (shown as a large arrow), the edge 31 of the central flange 33c neighboring the slot 311 can drag both ends 22, 22′ of the tubing 2 in this direction, and the tubing 2 can be retracted into the device 1.

In one exemplary embodiment, the casing 6 can further comprise a circumferential wall. In such a device 1, the spool 3 can fully be enclosed in the casing 6.

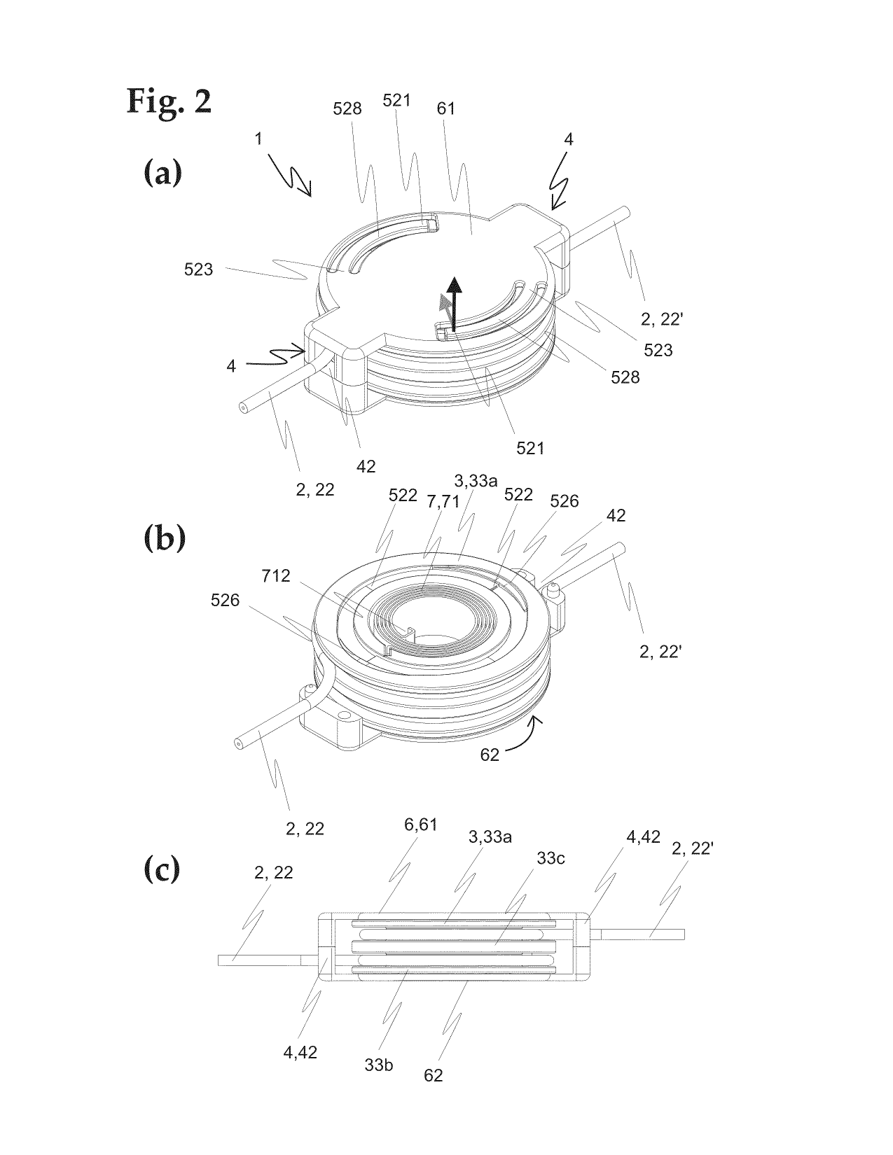

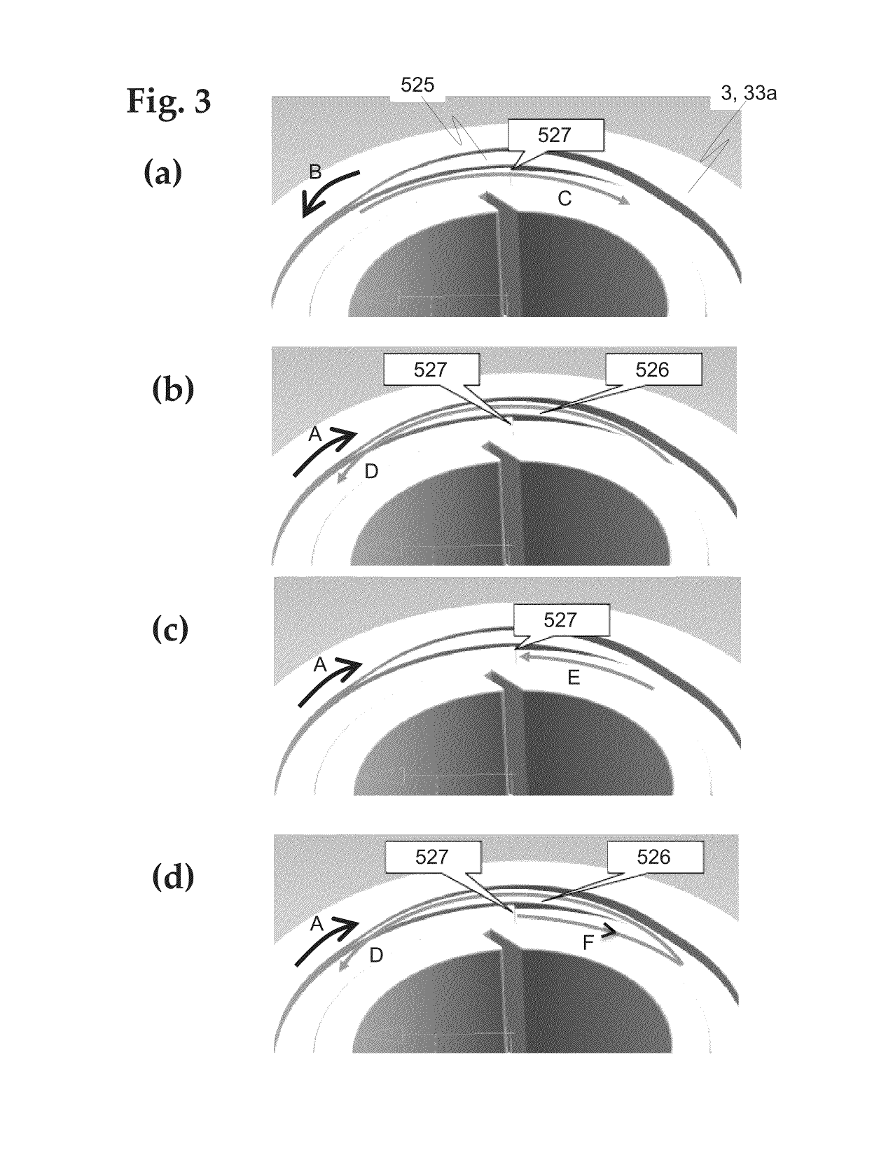

FIG. 2 shows another embodiment of a device 1, also with automatic retraction, (a) in a perspective view, (b) in a perspective view with removed top cover 61, and (c) in a side view. FIG. 2 differs from the device 1 in FIG. 1 essentially by its locking mechanism. In FIG. 1, a ratchet mechanism 52 controlled by a button 51 can be used to stop the retracting motion of the spool 3 driven by the spring coil 71, the embodiment in FIG. 2 uses a self-locking and unlocking ratchet mechanism 52, shown in more detail in FIG. 3.

The spool 3 can comprise a ratchet gear 524 with four teeth 522 arranged on one of the outer delimiting flanges 33a. Two paws 521 can be formed as an integral part of the top cover 61. The paws 521 can be connected to the cover 61 by long necks 528, the connection between the neck 528 and the cover 61 acting as a hinge area 523.

When a user pulls the ends 22, 22′ of the tubing 2 into opposite directions, the spool 3 can rotate in the releasing direction B, and the paws 521 can move along the path C on the ratchet gear 524, by jumping over the barriers 527 of the teeth 522 (see FIG. 3(a)). During this movement, each paw 521 can be bent out of the plane by the flat ramp of tooth 522, and can also be bent radially inwards by the barrier of a second, reversed ramp 526. The user thus can increase the length of the tubing 2 by pulling it out of the device 1. When the user stops pulling the tubing 2, the spool 3 can start retract the tubing 2, actuated by the biased spring coil 71. The paws 521 can follow a path D over the reverse ramp 526 and jump over the barrier of the reverse ramp (see FIG. 3(b)).

The user can then stop the tubing 2 from retracting and can pull it out far enough to let the paws 521 jump over the barrier 527 of teeth 522, but not far enough to let them reach the beginning of the reverse ramp 526. When the user releases the tubing 2 again, the spool 3 can be rotated by the coil 71 in the retraction direction A. However, since the paws 521 are not on the reverse ramps 526, each paw 521 can be bent radially inwards by the barrier of the ramp 526 and returns E to the barrier 527 of the tooth 522, where paws 521 and teeth 522 pairwise hook into each other and lock the ratchet mechanism 52 (see FIG. 3(c)).

To unlock the ratchet mechanism 52, the user pulls out the tubing 2 far enough that the paw 521 reaches F the beginning of the reverse ramp 526 (see FIG. 3(d)). When now the user releases the tubing 2 again, the spool 3 can again rotate unhindered in the retraction direction A, while the paws 521 follow path D along reverse ramp 526.

The device 1 disclosed in FIGS. 2 and 3 can have the advantage that no button has to be pressed by a user, which may be difficult when the device 1 is placed on something flexible such as, for example, on the abdomen of a user. The user may release and retract the tubing 2 according to his wishes, and lock and unlock the retraction function, just by simply increasing and decreasing the pulling force on the tubing 2.

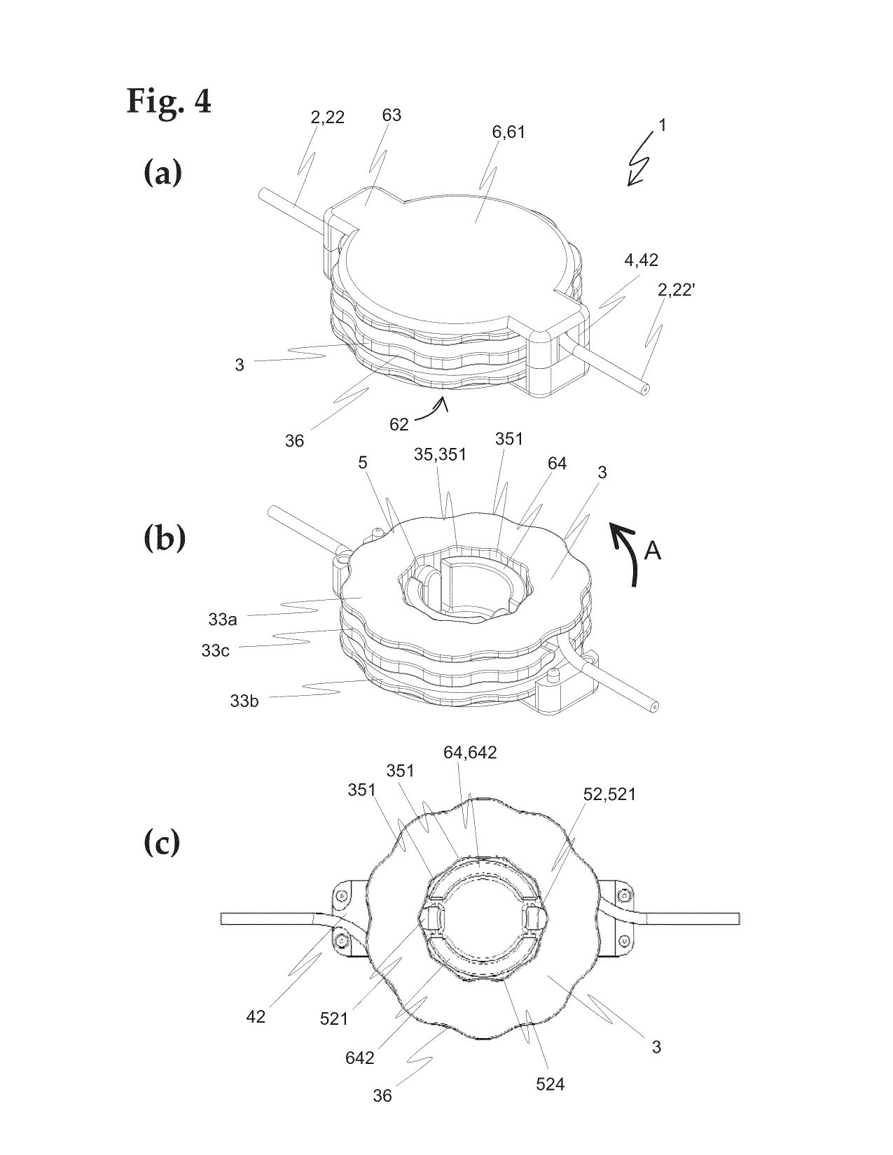

Again, the casing 6 of the device 1 of FIG. 2 may comprise a circumferential wall, protecting the content of the spool 3 from mechanical damage. Both embodiments in FIGS. 1 and 2 may also be realized with a single spool 3 compartment, as is shown in FIGS. 3 and 4.

In another embodiment with ratchet-like locking mechanism, the ratchet mechanism 52 can be realized as part of the bore 35 of the spool 3 and the hub 64 on which the spool 3 is mounted. Similarly, the spring coil 71 alternatively can be arranged between an outer delimit flange 33a, 33b and a cover of the casing 6.

Other advantageous embodiments of a device 1 can be realized fully manually operated, without the need for a retracting spring coil 71 or a similar actuation mechanism. An example of such an embodiment is depicted in FIG. 4, (a) in a perspective view, (b) in a perspective view with removed top cover 61, and (c) in a top view with removed top cover 61.

The device 1 can comprise a casing 6 with top cover 61 and bottom cover 62, in which the spool 3 is rotatably mounted on a hub 64 protruding from the inner side of the bottom cover 62. The spool 3 can be realized with two compartments, similar to FIGS. 1 and 2, and also the guiding structures 4 can be the same. To release the tubing 2 from the device 1, the user can pull the ends 22, 22′ of the tubing 2. To retract the tubing 2 into the device 1, the user can manually turn the spool 3 along the retraction direction A. For that purpose, one or more of the outer edges of the delimiting flanges 33a, 33b, 33c can be provided with suitable grip 36. In one example, the edges can be provided with depressions for better grip. Other possibilities can be, for example, a knurling on the edges of the spool 3, or a coating with a suitable material for increased grip such as, for example, a soft, rubber-like polymer.

As in the previously discussed embodiments, the device 1 can be provided with a ratchet-like 52 locking mechanism 5. For that purpose, the hub 64 can be subdivided into two guiding components 642, both having essentially the form of a half circle, and two paws 521 protruding perpendicularly from the bottom cover 62 and being arranged between the guiding structures 642. The essentially cylindrical bore 35 of the spool can have a corrugated surface, with a number of axially oriented grooves 351. In one exemplary example, there can be ten grooves. The outer diameter of the hub guiding structures 642 can correspond to the minimum diameter of the bore 35 between the grooves. The outer distance of the paws 521, on the other hand, can correspond to the maximum diameter of the bore 35 measured between two opposite grooves.

If a user pulls the tubing 2, thereby rotating the spool 3 in the release direction B, or manually turns the spool 3 in the retraction direction A, the two paws 521 of the ratchet mechanism 52 can be bent inwards by the convex area between the grooves 351 of the bore 35 and can engage again with the neighbouring grooves 351. This deformation of the paw 521 can require a certain force. Thus the spool 3 can immediately stop its rotation in regard to the casing 6 when the tubing 2 or the spool 3 is released by the user, and the tubing 2 cannot accidentally unwind itself.

An advantage of the device 1 shown in FIG. 4 can be that the ratchet mechanism 52 can unlock itself if the pulling force on the tubing 2 exceeds a certain threshold. If a user accidentally hooks the tubing 2 in an obstacle when moving, for example, a door knob, the ratchet mechanism 52 can unlock and tubing 2 will be released. This can prevent the infusion needle from being ripped out of the body of the user, which can be rather painful. Such a device 1 thus can also act as a security measure to prevent user injuries. Furthermore, the disclosed ratchet-type mechanism 52 can allow a user to precisely control the retraction and release of the tubing 2, which can be a great advantage, compared to the example of simple frictional locking of the spool 3 on the hub 64. Alternatively, the paws 521 can be arranged in the wall of the bore 35 and the hub 64 can be provided with grooves.

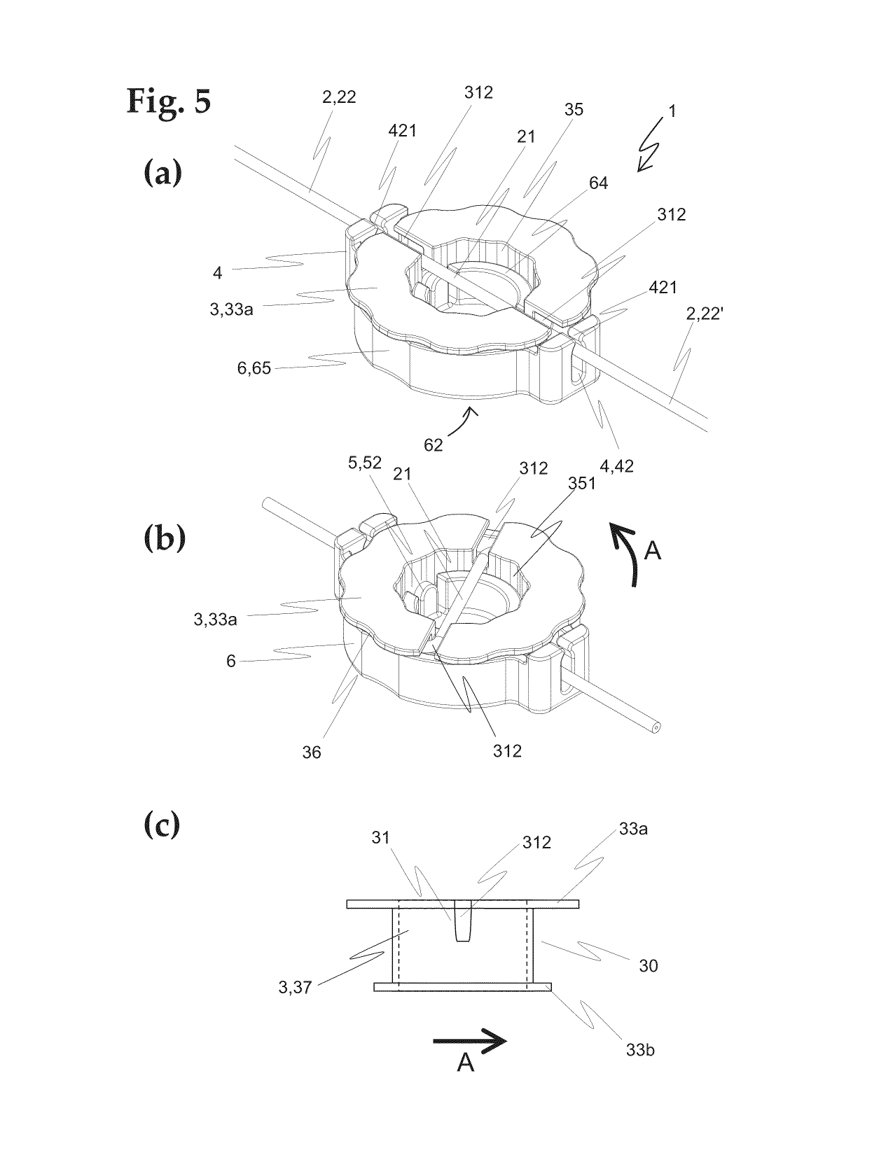

Another manually operated embodiment of a device 1 is depicted in FIG. 5. In this embodiment, the tubing 2 can be inserted without disconnecting its ends 22, 22′, for example, from the infusion cannula or the infusion pump.

FIG. 5 shows (a) a perspective view of the device 1 directly after threading the tubing 2 into the device 1, (b) a perspective view of the device 1 after a rotation of the spool 3 of about 105° along the retraction direction A, and (c) a side view of the spool 3.

The device 1 can comprise a casing 6 and a spool 3. The casing 6 can comprise a bottom cover 62, a circumferential wall 65 and two guiding structures 4, protruding from the wall 65. The spool 3 can comprise two outer delimiting flanges 33a, 33b, defining one single tubing compartment. The spool 3 can be rotatably mounted on the casing 6, with the bore 35 of the spool 3 being arranged on the hub 64 attached to the inner side of the bottom cover 62, similar to the embodiment in FIG. 4. A ratchet mechanism 52 similar to FIG. 4 can be used as the locking mechanism 5 of the device 1. To allow the manual rotation of the spool 3 by the user, the upper flange 33a can extend over the wall 65 and can be provided with a grip structure 36.

Since the casing 6 does not comprise a top cover 61, the spool 3 cannot be positively locked between two covers 61, 62. Thus the spool 3 not only has to be locked in its rotational movement on the hub 64, but also its position on the hub axis has to be secured. For that purpose, the grooves 351 on the wall of the bore 35 can extend in the axial direction only over a central part of the bore 35. When the spool 3 is placed on the hub 64, the paws 521 of the ratchet mechanism 52 can snap into the grooves, thereby locking the spool 3 on the hub 64 in the axial direction.

To allow the threading of the fluidly connected tubing 2 into the device 1, the openings 42 of the guiding structures 4 can have slots 421 toward the upper side of the device 1. Two opposite slots 312 can extend over the upper flange 33a of the spool 3 and an upper portion of the spool body 37. The width of the slots 312 can be equal or wider than the diameter of the tubing 2. The width of the slots 421 of the openings 42 can be preferably slightly smaller than the tubing 2, to prevent the tubing 2 from leaving the opening 42. To insert the tubing 2 into the device 1, the slots 312 of the spool 3 and the slots 421 of the guiding structure 4 can be aligned. The user now can insert the tubing 2 into the slots 312, 421, as shown in FIG. 5(a). Since the slots of the guiding structure can be narrower than the tubing 2, it can temporarily be compressed during insertion. The tubing 2 can now run through the openings 42 of the guiding structures 4 and the slots 312 of the spool body 37.

With the tubing 2 inserted, in a next step the user can rotate the spool 3 in a retracting direction A. The edges 31 of the slots 312 can drag the tubing 2 along the direction of rotation, and both ends 22, 22′ of the tubing 2 can be retracted into the device 1 and wound in the spool compartment 30.

Since the device 1 of FIG. 5 can be attached and removed from the tubing 2 at any time without disconnecting the tubing 2, it can be used in a very flexible manner. During the night, a user may place the infusion pump device 1 on his bedside table. The pump can be fluidly connected to the infusion cannula by a tubing 2. The length of the tubing 2 can be sufficient to take into account any movements of the user during sleep. After getting up, the user can attach the pump device 1 to his body or his clothes. He can then thread the tubing 2 into the device 1 or mount the device 1 to the tubing 2, respectively. He can then turn the spool 3 to retract the surplus tubing 2 as far as necessary and attach the device 1 to his body or his clothes.

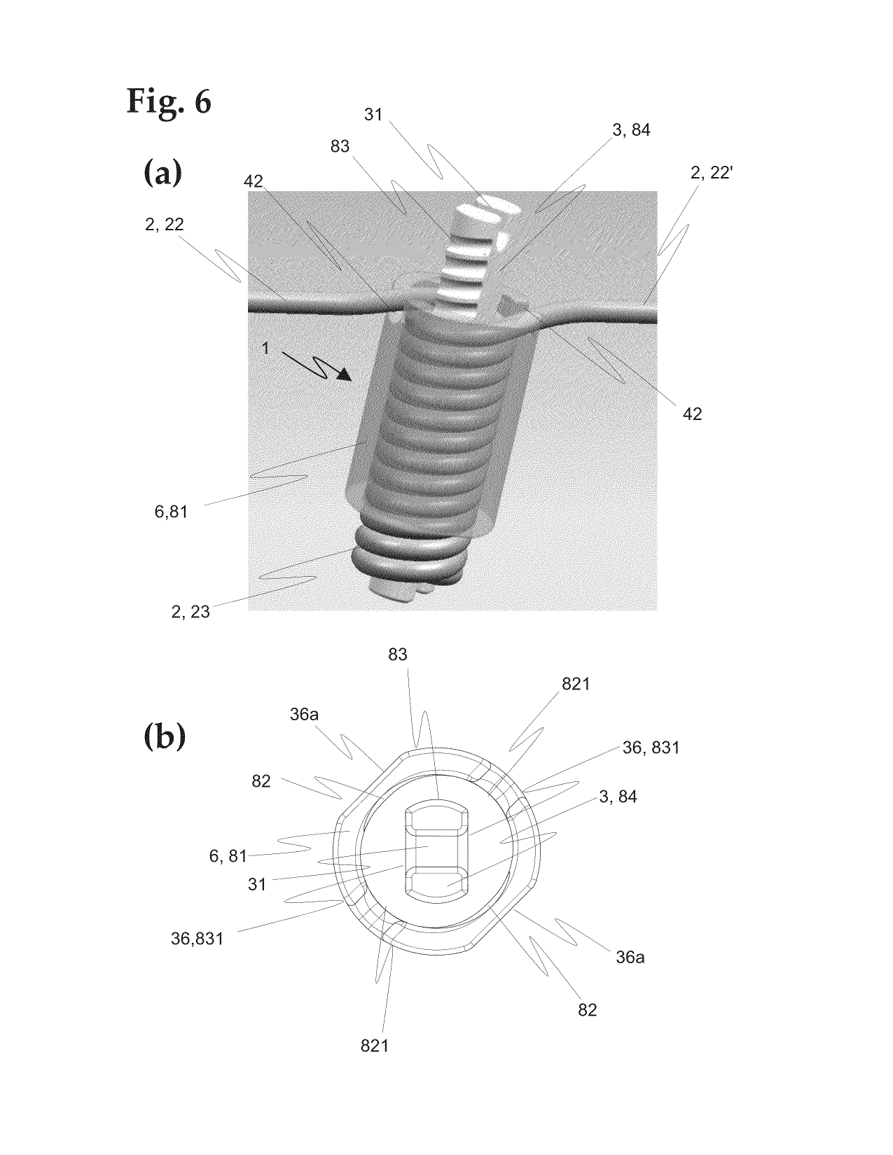

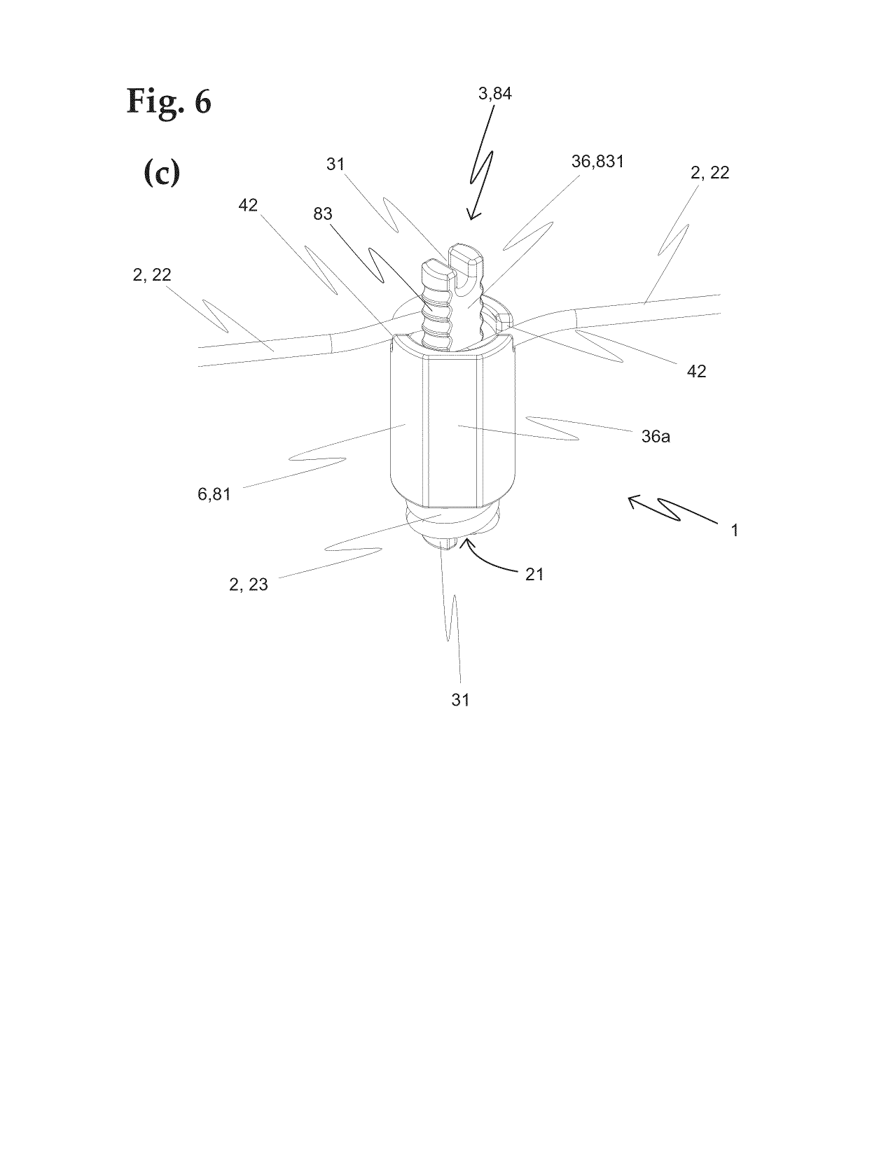

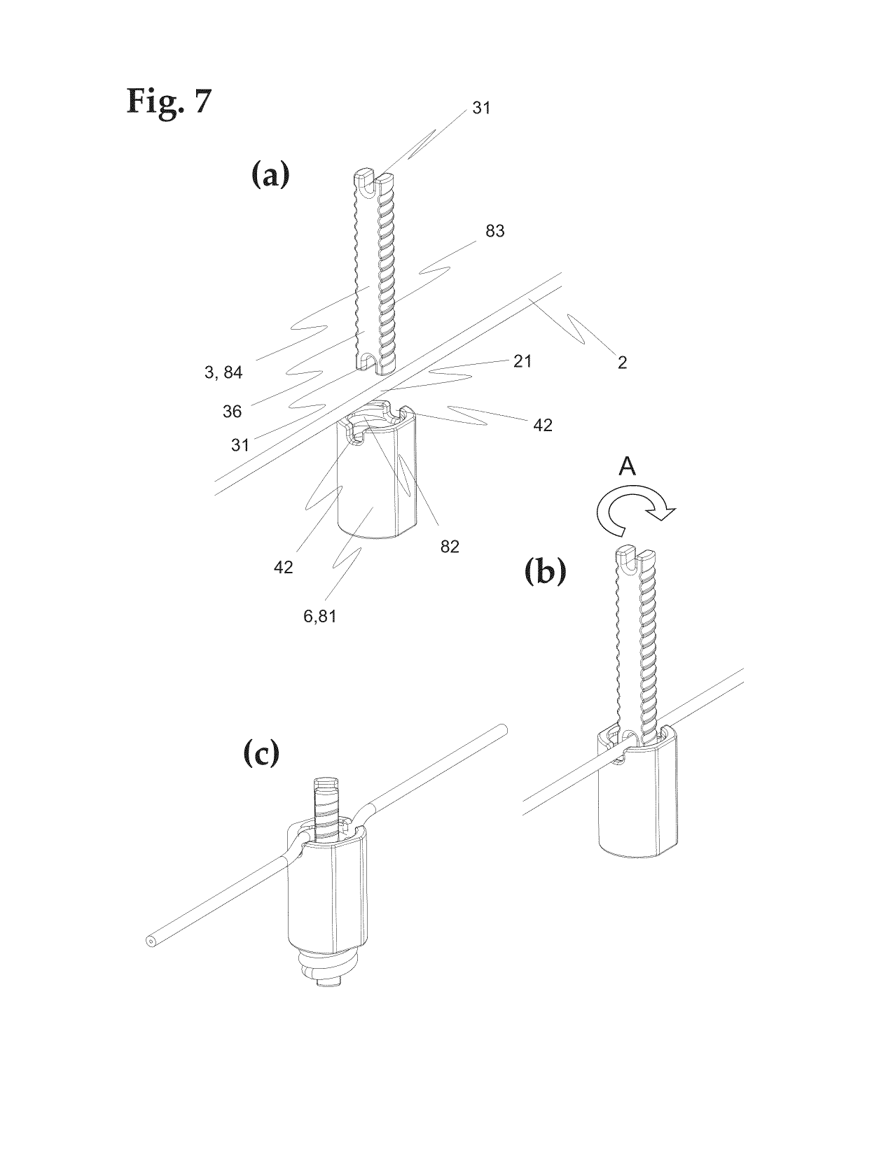

Yet another embodiment of a device 1 is shown in FIG. 6 (a) and (c) in a perspective view with wound up tubing 2, and (b) a top view without tubing 2. In FIG. 6(a) a part of the device 1 is shown partially transparent. FIG. 7 (a) shows the device 1 (c) before assembly, (b) prior to winding up the tubing 2, and (c) after winding up the tubing 2.

This embodiment of a device 1 can comprise a spindle-like spool 3 as an inner unit 84 and a casing 6 as an outer unit 81. In the device, the inner and outer unit 84, 81 can embrace the coiled surplus tubing 23. The inner unit 3, 84 can essentially be a cylinder with outer thread 83, of which along two opposite sides the thread can be removed 831, resulting in two flat areas 36, where a user can hold the inner unit 84. Both ends of the inner unit 84 can have a notch 31 for holding the tubing 2.

The outer unit 6, 81 can essentially be a hollow cylinder, which can have an inner thread 82 on two opposite sides on the inner surface and two areas 821 without thread. The outer unit 3, 84 can further comprise two flat areas 36a on the mantle for better grip. The inner thread of the outer element is not essential for the function of the device 1.

With the tubing 2 wound up to the device 1, the coiled tubing 23 facing the inner unit 84 can interact with the outer thread 83 and the inner thread 82 can interact with the coiled tubing 23 facing the outer unit 81. Thus the coiled tubing 23 can act as the counter thread for the inner thread 82 of the outer unit 81 and as the counter thread of the outer thread 83 of the inner unit 84. The tubing 2 can be wound up as a double helix, a central portion 21 being held in one of the slots 31.

For winding up the tubing 2 on the device 1, the tubing 2 can be placed in two opposite notches 42 on the circumference of the outer unit 81, and the notch 31 of the inner unit 84 can also be placed on the tubing 2, both units 81, 84 being aligned (see FIG. 7(b)). While holding the outer unit 81, the user can rotate the inner unit 84 in a retracting direction A and can push it toward the outer unit. The notch 31 can force the tubing 2 to rotate, which consequently can form a tubing coil 23 around the outer threading 83. At the same time, the notches 42 can fix the tubing 2 in regard to the inner thread 82. Thus by rotating the inner unit 3, 84 the tubing 2 can form a coil 23 that can screw itself and the inner unit 84 into the thread 82 of the outer unit 81, at the same time retracting the tubing 2 into the device 1. For releasing the tubing 2, the inner unit can be turned into the opposite direction, thereby reversing the process.

Since both for the inner thread 82 and outer thread 83 the thread can be removed on two opposite sides, the tubing coil 23 can be slightly compressed between the threaded portions when a portion of the inner thread 82 passes a portion of the outer thread 83. This can result in a resilient force that can be repeatedly overcome by the user when rotating the inner unit with the coil 23. Thus the inner unit, the tubing coil 23, and the outer unit can form a ratchet-like locking mechanism.

The device as shown in FIGS. 6 and 7 can be attached to the already connected tubing 2 and removed without disconnecting the tubing 2. Due to its oblong form, it can be space-saving, while at the same time, storing a considerably amount of tubing 2. The device 1 can be produced at very low costs, since it comprises only two parts having a geometry that is easy to produce, for example, with injection moulding. The device 1 can be produced by injection moulding using suitable polymer materials. The dimensions of the device 1 can be given by the length and diameter of the tubing 2 to be stored.

The device 1 according to the present disclosure may be fastened to the body by adhesive tape, to the belt or the clothes by using a suitable clip, or hook-and-loop fastener (Velcro®). It may also be realized as an integral part of a pump casing 6.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A device for storing and dispensing infusion tubing, the device comprising:
a casing;
a spool for storing the infusion tubing, rotatably mounted on the casing; and
a locking structure for positively locking a central portion of infusion tubing on the spool by a locking mechanism suitable for locking a rotation of the spool in regard to the casing in at least one direction.

2. The device according to claim 1, wherein the locking mechanism can lock the rotation of the spool in two directions.

3. The device according to claim 1, further comprising, guiding structures for the infusion tubing.

4. The device according to claim 1, wherein the locking mechanism is a ratchet mechanism comprising a ratchet gear comprising a plurality of circularly arranged grooves, or teeth, and one or more paws interacting with the ratchet gear.

5. The device according to claim 4, wherein the ratchet gear is arranged on an outer delimiting flange of the spool and the paws are attached to the casing.

6. The device according to claim 4, wherein the paws are arranged on an outer delimiting flange of the spool and the ratchet gear is attached to the casing.

7. The device according to claim 1, wherein the spool comprises a bore rotatably mounted on a hub attached to the casing

8. The device according to claim 7, wherein the locking mechanism is a ratchet mechanism comprising a ratchet gear consisting of a multitude of circularly arranged grooves, or teeth, and one or more paws interacting with the ratchet gear, wherein the ratchet gear is arranged on the circumference of the bore and the paws are arranged on the hub.

9. The device according to claim 7, wherein the locking mechanism is a ratchet mechanism comprising a ratchet gear consisting of a multitude of circularly arranged grooves, or teeth, and one or more paws interacting with the ratchet gear, wherein the paws are arranged on the circumference of the bore and the ratchet gear is arranged on the hub.

10. The device according to claim 1, further comprising, an actuation mechanism for rotating the spool in one direction.

11. The device according to claim 10, wherein the actuation mechanism is a spring coil.

12. The device according to claim 1, wherein an outer delimiting flange of the spool comprises two radial slots on opposite sides of the flange extending to axial slots in the spool body.

13. The device according to claim 12, wherein guiding structures for the infusion tubing, with openings through which the infusion tubing can run to the spool and wherein the openings comprise slots facing toward the same direction as the radial slots.

14. The device according to claim 1, wherein the spool is an inner unit in the form of a cylinder, having an outer thread and two or more areas without threading and wherein the casing is an outer unit in the form of a cylinder barrel, wherein the dimensions of the inner and outer unit and the outer thread are chosen in such a way that infusion tubing with a certain diameter can be wound up to the inner unit, forming a tubing coil, and that the tubing coil can be arranged in the outer unit.

15. The device according to claim 14, wherein the outer unit has an inner thread and two or more areas without threading, wherein the dimensions of the inner and outer unit and the threads are chosen in such a way that infusion tubing with a certain diameter can be wound up to the inner unit, forming a tubing coil, and that the tubing coil can be screwed into the thread of the outer unit.

16. A device according to any of claim 1, wherein the device has infusion tubing wound up on the spool.

17. The device according to claim 16, wherein the device and the infusion tubing are sterilized.

18. The device according to claim 16, wherein the infusion tubing comprises connectors on one or both ends for connecting the infusion tubing to an infusion pump device and/or an infusion cannula or needle.

19. A set comprising a device according to claim 1 and one or more pieces of infusion tubing.

20. Infusion tubing with one or more devices according to claim 1 attached to the infusion tubing.
